(19) United States
(12) Patent Application Publication
(10) Pub. No.: US 2024/0112778 A1
(43) Pub. Date: Apr. 4, 2024

(54) System For Enhancing Drug Treatment Of A Patient

(71) Applicant: GAIA AG, Hamburg (DE)

(72) Inventor: Mario Weiss, Hamburg (DE)

(21) Appl. No.: 17/937,275

(22) Filed: Sep. 30, 2022

(51) Int. Cl. G16H 20/10; A61B 5/00; A61B 5/1171

(52) U.S. Cl. CPC G16H 20/10; A61B 5/1176; A61B 5/7264; A61B 5/746

(57) ABSTRACT

A system for enhancing drug treatment of a patient is disclosed having a computer, a database in data communication with said computer, said database having a plurality of sensory prompts, software executing on said computer for querying the database for sensory prompts corresponding with treatment of the patient, a user device in data communication with said computer for presenting at least one retrieved sensory prompt, a sensor associated with said user device for collecting patient information in response to the sensory prompt, software executing on said computer for receiving the patient information, software executing on said computer for inducing an activity or thought process modification based the patient information, software executing on said computer for transmitting activity, sensory data and thought process modification to a medical provider.

### TECHNICAL FIELD

The present disclosure relates to a novel system for enhancing drug treatment of a patient. More specifically, the present disclosure relates to modern drugs which come with a digitalized service to enhance the benefit of the drugs and delivers a holistic outcome by aiding patients to modify lifestyle and thinking in a way to improve their treatment.

### BACKGROUND

An objective of internal medicine is to change the biological/chemical system of the patient in order to improve health. This is normally done with a molecule that comes into the body via a pill or an injection. By interacting with other molecules and receptors in the body, biological change may be induced to improve health. The additional pathway to change the system is to adapt behavior (e.g. eat healthier, do more exercise, take your pills) or thought process (e.g. reduce stress). Modifying behavior and thought process is an important part of treatment with impacts additive to drug therapy.

One way healthy behavioral change can be induced is by showing media—text, pictures, etc., to a patient. However, physicians normally do not have the right media at hand to stimulate the appropriate thought and behavioral change for each individual patient and drug therapy. Moreover, the interpretation of myriad behavioral and thought interventions upon drug treatment and medical outcome may be difficult for a physician to understand or interpret.

In addition, medical professionals normally specialize in drug treatment or behavioral change treatment, not both. Referrals are therefore required when a drug treatment patient requires or would benefit from behavioral change. These referrals increase treatment time and cost and may themselves exacerbate a patient's condition.

### SUMMARY

For these and other reasons known to a person of an ordinary skill in the art, what is needed is a system that allows patients to benefit from media designed to change behavior and/or thought processes.

Another goal of the present disclosure is to provide a system that enhances patient drug treatment outcomes.

Another goal of the present disclosures is to provide a system that can improve the effectiveness of drug treatments.

Another goal of the present disclosures is to provide a system that can manage the behavioral and thought process change part of treatment and could be prescribed by the physician responsible for drug therapy.

Another goal of the present disclosures is to provide a behavioral and thought process change system that can receive feedback and improve its effectiveness.

In one aspect of the present invention, a system for enhancing drug treatment of a patient is disclosed. The system has a computer and a database in data communication with said computer. The database has a plurality of sensory prompts. Software executing on said computer queries the database for sensory prompts corresponding with a particular drug treatment of the patient. A user device in data communication with said computer presents at least one retrieved sensory prompt. A sensor associated with said user device collects patient information in response to the sensory prompt, which is received by said computer. Software executing on said computer for recommends an activity or behavior modification based the patient information and transmits the recommended activity modification to a medical provider.

### BRIEF DESCRIPTION OF THE DRAWINGS

FIG. 1 shows a schematic diagram of the presently disclosed system.

### DETAILED DESCRIPTION

The present invention will now be described by referencing the appended FIGURE.

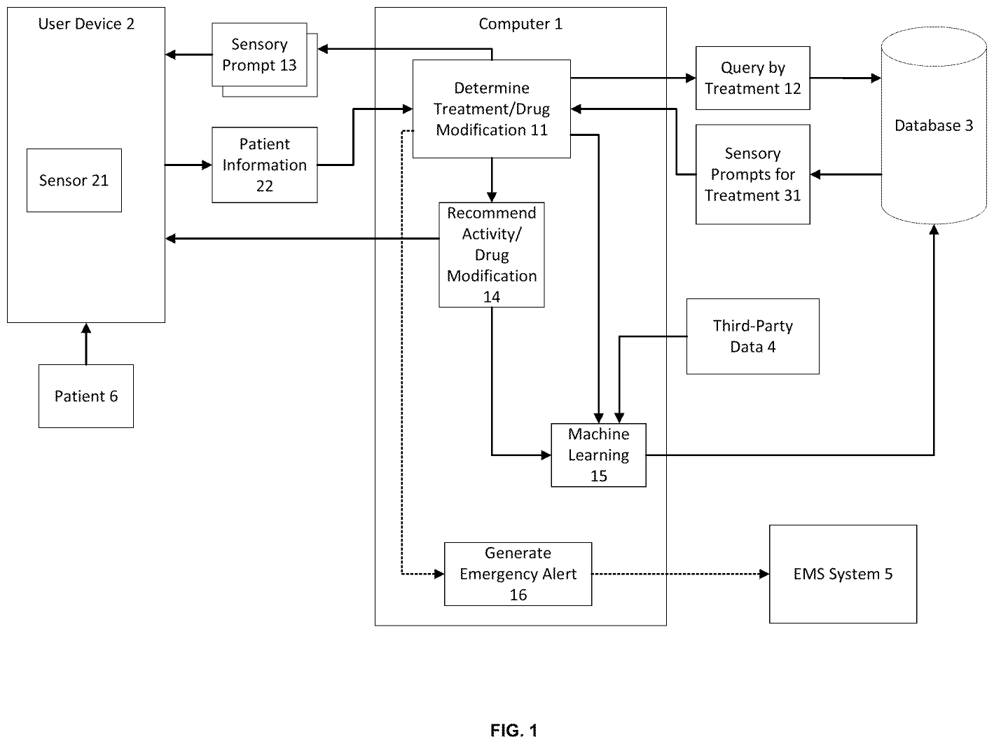

Referring to FIG. 1, the present disclosure describes a system 10 for enhancing drug treatment of a patient.

The system 10 includes a computer 1. The computer 1 may be a processor, remote computer, computer server, network, or any other computing resource, including mobile devices.

The computer 1 may be in data communication with a user device 2. The user device 2 may be a computer, laptop, smartphone, tablet, or other electronic device, including mobile devices, capable of transmitting data to the computer 1. User device 2 may run an application on a mobile device or smartphone. The user device 2 may have an input device such as a mouse and keyboard, touchscreen, trackpad, etc. The user device 2 may include a display.

The user device 2 may include a sensor 21. The sensor 21 may be a camera, microphone, heart rate sensors, blood pressure sensors, or temperature sensors. The sensor may capture data regarding a biosignal, such as heart rate variability, blood pressure, sleep deprivation or emotional stress. Specialized software may be required to analyze the sensor's output, for instance in the case of cameras, which may require computer vision software. The sensor 21 may be integrated in or external to the user device 2.

The computer 1 may also be in communication with a database 3. The database 3 may store information regarding the system 10, including queries 12 and sensory prompts 31. The database 3 may be a storage drive or array accessible to computer 1, or cloud storage. Sensory prompts 31 may be indexed or searchable by treatments. Treatments can be drugs, therapies, remedies, or the like, alone or in combination. The database 3 may receive queries 12 for sensory prompts 31 by treatment.

Prompts 31 may include any media that stimulates the patient 6. For example, prompts 31 may include text, images, sound, video, physical stimuli, tasting material, etc.

Queries 12 may seek a category of prompts 31 (e.g., treatment), or a specific prompt 31. Categories of prompts 12 may on the prompt itself, the type of prompt (image, sound, etc.), previous prompts or the patient's 6 reaction thereto, or categorizations based on conditions, medications, or other factors. For example, the query may specify that the patient has high blood pressure and is being treated with ACE inhibitors.

The computer 1 may include software 11 to determine a whether modified behavior and thinking has occurred and if so whether those changes have enhanced treatment of the patient 6. To that end, it may send a query 12 to the database 3 for a prompt 31. The query 12 may specify the drug treatment the patient 6 is undergoing. The prompt 31 may be sent to the user device 2 as prompt 13 after being processed by the software 11. The software 11 may process the prompt 31 by identifying an expected emotion in response to the prompt 31.

The user device 2 presents the prompt 13 to the patient 6. The patient 6 may experience a response in view of being presented with prompt 13. For instance, the blood pressure or temperature may rise. The sensor 21 may capture patient information 22 regarding the relevant metric before, during, and after the patient 6 is presented with the prompt 13. The user device 2 provides the patient information 22 to the computer 1. In addition, the patient 6 may provide additional feedback via the user device 2 as patient information 22.

The software 11 determines a recommended activity or thought process modification based on the patient's drug treatment and prompt response information 22. The software 11 may use a neural network to analyze and classify the patient response information to determine a recommended activity of thought process modification 14. The recommended activity thought modification 14 is designed to be additive to the patient's drug treatment in the sense that it activates the same cellular activity as the drug to enhance a patient's outcome (e.g., reduce stress so that a drug works more effectively). In our example, the patient taking ACE inhibitors could see an additive reduction in blood pressure by reducing stress in their life and getting additional exercise.

The computer may use third-party data 4 to help determine the recommended activity or thought process modification. Third party data 4 may include the weather at location of patient 6, potential stressors such as crime rate (communicated crime in media), pollution, traffic (time spend in traffic), and psycho economics such as stock price, inflation rate, employment rate (specifically in the sector patient 6 is working in), and consumer index. For example, if it is raining outside, the system may not recommend an outdoor activity.

The recommended activity or thought process modification 14 may be provided to the user device 2. The recommended activity or thought process modification 14 may be a new sensory prompt 13 that is shown to the patient 6 via user device 2. The prompt 13 may be designed to induce happiness, for example. Alternatively, the recommended activity or thought process modification 14 may be a suggested activity to the patient 6, such as twenty minutes of exercise. As another example, a recommended activity modification 14 may also be a new prescription to help deal with side effects of a treatment the patient 6 is already undergoing.

The computer 1 may receive additional patient response information 22 corresponding to the act of the patient 6 performing or having recently performed the recommended activity or thought process modification 14. Such information may be used by the software 11 to determine a new recommended activity or thought process modification 14.

The computer may use any of the prompts 13, patient information 22, the results of the drug treatment enhancement software 11 (e.g., recommended activity or thought process modification 14), additional patient information 22, and third-party data 4 to perform machine learning 16 and improve its functionality. For example, the computer 1 may refine determine treatment modification software 11 using this information. Database 3 may store the results of the machine learning process 16. In this way, the system can learn from its continued use.

The system may run in the background and not interfere with other treatments or activities. In such situations, the system may constantly refine its determination a recommended activity modification.

If provided patient information 22 is potentially harmful or dangerous, the computer 1 may generate an emergency alert 17. The alert may be communicated to an EMS system 5, a designated contract, medical professional, or other person.

Although the invention has been illustrated and described herein with reference to a preferred embodiment and a specific example thereof, it will be readily apparent to those of ordinary skill that the art that other embodiments and examples may perform similar functions and/or achieve user experiences. All such equivalent embodiments and examples are within the spirit and scope of the present invention, are contemplated thereby, and are intended to be covered by the following claims.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect. The present disclosure is to be considered as an example of the invention, and is not intended to limit the invention to a specific embodiment illustrated by the FIGURES above or description below.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc. for different features/components of the present disclosure are only intended to distinguish the features/components from other similar features/components and not to impart any order or hierarchy to the features/components. As used herein, the term “and/or” includes any and all combinations of one or more of the associated listed items. As used herein, the term “application” is intended to be interchangeable with the term “invention”, unless context clearly indicates otherwise.

To aid the Patent Office and any readers of any patent issued on this application in interpreting the claims appended hereto, Applicant that it does not intend any of the claims or claim elements to invoke 35 U.S.C. 112(f) unless the words “means for” or “step for” are explicitly used in the particular claim.

While the present teachings have been described above in terms of specific embodiments, it is to be understood that they are not limited to these disclosed embodiments. Many modifications and other embodiments will come to mind to those skilled in the art to which this pertains, and which are intended to be and are covered by both this disclosure and the appended claims. It is intended that the scope of the present teachings should be determined by proper interpretation and construction of the appended claims and their legal equivalents, as understood by those of skill in the art relying upon the disclosure in this specification and the attached drawings. In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

What is claimed is:

1. A system for enhancing drug treatment of a patient comprising:
a computer;
a database in data communication with said computer, said database having a plurality of sensory prompts;
software executing on said computer for querying the database for sensory prompts corresponding with a drug treatment of the patient;
a user device in data communication with said computer for presenting at least one retrieved sensory prompt;
a sensor associated with said user device for collecting patient information in response to the sensory prompt;
software executing on said computer for receiving the patient information;
software executing on said computer for recommending an activity modification based the patient information;
software executing on said computer for transmitting the recommended activity modification to a medical provider.

2. The system of claim 1, wherein the activity modification is a thought process change.

3. The system of claim 1, wherein the activity modification is a physical activity change.

4. The system of claim 1, further comprising software executing on said computer for generating an emergency alert in response to the patient information.

5. The system of claim 1, further comprising software executing on said computer for training a machine learning algorithm based on at least one of the patient information and the recommended activity modification.

6. The system of claim 1 wherein said sensor collects biometric data from the patient.

7. The system of claim 6, wherein the biometric data is data regarding heart rate variability, blood pressure, sleep deprivation or emotional stress.

8. The system of claim 1, wherein the sensor is one of a microphone, heart rate sensor, blood pressure sensor, or temperature sensor.

9. The system of claim 1, wherein the sensor is a camera and the biometric data is an image of the patient's face.

10. The system of claim 1, wherein the activity modification is an additional prompt displayed to the patient.

11. A system for supplementing treatment of a patient comprising:
a computer;
a database in data communication with said computer, said database having a plurality of sensory prompts;
software executing on said computer for querying the database for sensory prompts corresponding with a medication of the patient;
a user device in data communication with said computer for presenting at least one retrieved sensory prompt;
a sensor associated with said user device for collecting patient information in response to the sensory prompt;
software executing on said computer for receiving the patient information;
software executing on said computer for providing a second sensor prompt based the patient information;
the sensor collecting a second patient information in response to the second sensory prompt;
software executing on said computer for receiving the second patient information.

12. The system of claim 11, further comprising software executing on said computer training a machine learning algorithm based on at least one of the patient information and the second patient information.

13. The system of claim 11, further comprising software executing on said computer for generating an emergency alert in response to the patient information.

14. The system of claim 11, further comprising software executing on said computer for training a machine learning algorithm based on at least one of the patient information and the recommended activity modification.

15. The system of claim 11, wherein the sensor is one of a camera, microphone, heart rate sensor, blood pressure sensor, or temperature sensor.

16. The system of claim 11, wherein said sensor collects biometric data from the patient.

17. The system of claim 16, wherein the biometric data is an image, or data regarding heart rate variability, blood pressure, or emotional stress.
